(19) United States
(12) Patent Application Publication
(10) Pub. No.: US 2022/0242880 A1
(43) Pub. Date: Aug. 4, 2022

(54) PROCESS FOR PREPARATION OF MIDOSTAURIN

(71) Applicant: DR. REDDY?S LABORATORIES LIMITED, Hyderabad (IN)

(72) Inventors: Swapna AKULA, Hyderabad (IN); Shravan Kumar KOMATI, Kattangur (IN); Siva Reddy MAKIREDDY, Sattenapalli (Mandal) (IN); Rajeev Rehani BUDHDEV, Hyderabad (IN); Sekhar Munaswamy NARIYAM, Hyderabad (IN); Lokeswara Rao MADIVADA, Mopidevi Mandal (IN)

(21) Appl. No.: 17/613,202

(22) PCT Filed: Jun. 23, 2020
(86) PCT No.: PCT/IN2020/050547

§ 371 Date: Nov. 22, 2021

(30) Foreign Application Priority Data
Jun. 24, 2019 (IN) 201941025028
Nov. 18, 2019 (IN) 201941046926

(51) Int. Cl. C07D 498/22

(52) U.S. Cl. CPC C07D 498/22

(57) ABSTRACT

The present application relates to a process for the preparation of midostaurin by controlling critical impurities or by-products which in turn lead to increase in the overall yield and purity. The present application also provides midostaurin having less than 0.15% or substantially free or free of one or more impurities.

### INTRODUCTION

Aspects of the present application relate to process for preparation of Midostaurin.

The drug compound having the adopted name midostaurin, is a semi-synthetic derivative of staurosporine chemically designated as N-[(2S,3R,4R,6R)-3-methoxy-2-methyl-16-oxo-29-oxa-1,7,17-triazaoctacyclo[12.12.2.1^{2,6}.0^{7,28}.0^{8,13}.0^{15,19}.0^{20,27}.0^{21,26}]nonacosa-8,10,12,14,19,21,23,25,27-nonaen-4-yl]-N-methylbenzamide and is represented by structure of formula I.

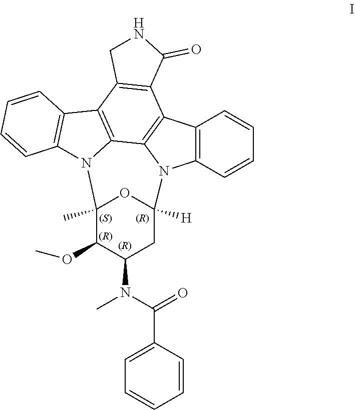

Midostaurin is a kinase inhibitor indicated for the treatment of adult patients with acute myeloid leukemia (AML), aggressive systemic mastocytosis (ASM), systemic mastocytosis with associated hematological neoplasm (SM-AHN), or mast cell leukemia (MCL).

U.S. Pat. No. 5,093,330 discloses midostaurin and process for its preparation.

U.S. Pat. No. 8,198,435 (435 patent) discloses crystalline form II, essentially amorphous, amorphous form of midostaurin and process for their preparation. The '435 patent also discloses purification process of staurosporine.

U.S. Pat. No. 9,150,589 discloses crystalline form III of Midostaurin and process for its preparation. U.S. Pat. No. 9,593,130 discloses crystalline form IV of Midostaurin and process for its preparation.

PCT publication No. WO2018/165071A1 discloses the various crystalline forms of Midostaurin and processes for their preparation. The said PCT application also discloses the purification of crude midostaurin using column chromatography.

The reported processes for preparation of midostaurin suffer from disadvantages like incomplete conversion reaction, formation of multiple impurities or unwanted by-products. Some of the impurities are known to be unusually potent or to produce toxic or unexpected pharmacological effects. The US Food and Drug Administration (FDA) as well as European Medicines Agency guidance suggest that the API is free from impurities to the maximum possible extent. To remove the impurities or unwanted by-products various purification steps or purification techniques are introduced which in turn leads to the decrease in the overall yield and increase in the operation expenses.

The present application provides a process for the preparation of midostaurin by controlling critical impurities or by-products which in turn lead to increase in the overall yield and purity.

### SUMMARY

In the first embodiment, the present application provides a process for preparation of midostaurin, said process comprising reacting staurosporine with benzoic anhydride in a solvent comprising halogenated hydrocarbon or mixture of halogenated hydrocarbon & alcohol to produce midostaurin.

In the second embodiment, the present application provides a process for preparation of midostaurin, said process comprising:

(a) reacting staurosporine with benzoic anhydride in a solvent comprising halogenated hydrocarbon or mixture of halogenated hydrocarbon & alcohol to produce midostaurin,

(b) isolating the midostaurin.

(c) optionally purifying the midostaurin obtained in step (b)

In the third embodiment, the present application provides a process for preparation of midostaurin, said process comprising:

(a) reacting staurosporine with benzoic anhydride in a solvent comprising mixture of halogenated hydrocarbon & alcohol to produce midostaurin,

(b) removing the halogenated hydrocarbon solvent,

(c) isolating the midostaurin, and

(d) optionally purifying the midostaurin obtained in step (c)

In the fourth embodiment, the present application provides midostaurin having less than 0.1% of one or more impurities selected from palmitoyl staurosporine, O-desmethyl midostaurin, N-desmethyl midostaurin, 7-oxo midostaurin, staurosporine, hydroxy midostaurin or N-benzoyloxy midostaurin.

In the fifth embodiment, the present application provides midostaurin free of one or more impurities selected from palmitoyl staurosporine, O-desmethyl midostaurin, N-desmethyl midostaurin or 7-oxo midostaurin.

In the sixth embodiment, the present application provides midostaurin substantially free of one or more impurities selected from staurosporine, hydroxy midostaurin or N-benzoyloxy midostaurin.

In the seventh embodiment, the present application provides process for preparation of midostaurin having less than 0.1% or free or substantially free of one or more impurities selected from palmitoyl staurosporine, O-desmethyl midostaurin, N-desmethyl midostaurin, 7-oxo midostaurin, staurosporine, hydroxy midostaurin or N-benzoyloxy midostaurin, said process comprising:

(a) reacting staurosporine with benzoic anhydride in a solvent comprising halogenated hydrocarbon or mixture of halogenated hydrocarbon & alcohol to produce midostaurin,

(b) isolating the midostaurin.

(c) optionally purifying the midostaurin obtained in step (b)

In the eighth embodiment, the present application provides substantially pure midostaurin.

### DETAILED DESCRIPTION

In the first embodiment, the present application provides a process for preparation of midostaurin, said process comprising reacting staurosporine with benzoic anhydride in a solvent comprising halogenated hydrocarbon or mixture of halogenated hydrocarbon & alcohol to produce midostaurin.

In the second embodiment, the present application provides a process for preparation of midostaurin, said process comprising:

(a) reacting staurosporine with benzoic anhydride in a solvent comprising halogenated hydrocarbon or mixture of halogenated hydrocarbon & alcohol to produce midostaurin,

(b) isolating the midostaurin.

(c) optionally purifying the midostaurin obtained in step (b)

Step (a) involves reacting staurosporine with benzoic anhydride in a solvent comprising halogenated hydrocarbon or mixture of halogenated hydrocarbon & alcohol to produce midostaurin

The step (a) may be carried out at a temperature less than 100° C. or less than 80° C. or less than 60° C. or less than 40° C. or less than 20° C. or at the reflux temperature of the solvent or mixture of solvents used in step (a).

Step (b) involves isolating midostaurin.

Suitable isolation methods that may be used in step (b) include decantation or filtration or precipitation from a solvent or by removing the solvent or by concentrating the reaction mass or adding an anti-solvent to a solution or by evaporation of solution and the like or any other suitable isolation techniques known in the art. The said precipitation may result in a crystalline compound including solvates and hydrates thereof or amorphous form or essentially amorphous form. Suitable solvents that may be used for said isolation include water, alcohols, ketones, hydrocarbons, halogenated hydrocarbons, esters, ethers, polar aprotic solvents, nitriles or any mixtures thereof.

Step (c) involves optionally purifying the midostaurin obtained in step (b)

Purification of midostaurin obtained from step (b) may be carried out by one or more methods selected from slurrying in a solvent, recrystallization from a solvent or a chromatography.

Suitable solvents that may be used for purification of midostaurin by slurrying in a suitable solvent or recrystallization in a solvent include water, alcohols, ketones, hydrocarbons, halogenated hydrocarbons, esters, ethers, polar aprotic solvents, nitriles or any mixtures thereof.

Suitable chromatographic techniques that may be used for purification of midostaurin are selected from column chromatography, flash chromatography, ion exchange chromatography, supercritical fluid chromatography, high performance liquid chromatography (both reverse phase and normal phase), expanded bed adsorption chromatography and simulated moving bed chromatography or any combination thereof.

The purification process may be carried out one or more times using one or more purification methods described in the present application to completely remove the impurities or to get the desired purity of midostaurin.

In the third embodiment, the present application provides a process for preparation of midostaurin, said process comprising:

(a) reacting staurosporine with benzoic anhydride in a solvent comprising mixture of halogenated hydrocarbon & alcohol to produce midostaurin,

(b) removing the halogenated hydrocarbon solvent,

(c) isolating the midostaurin, and

(d) optionally purifying the midostaurin obtained in step (c)

In the fourth embodiment, the present application provides midostaurin having less than 0.1% of one or more impurities selected from palmitoyl staurosporine, 0-desmethyl midostaurin, N-desmethyl midostaurin, 7-oxo midostaurin, staurosporine, hydroxy midostaurin or N-benzoyloxy midostaurin.

In the fifth embodiment, the present application provides midostaurin free of one or more impurities selected from palmitoyl staurosporine, 0-desmethyl midostaurin, N-desmethyl midostaurin or 7-oxo midostaurin.

In the sixth embodiment, the present application provides midostaurin substantially free of one or more impurities selected from staurosporine, hydroxy midostaurin or N-benzoyloxy midostaurin.

In the seventh embodiment, the present application provides process for preparation of midostaurin having less than 0.1% or free or substantially free of one or more impurities selected from palmitoyl staurosporine, 0-desmethyl midostaurin, N-desmethyl midostaurin, 7-oxo midostaurin, staurosporine, hydroxy midostaurin or N-benzoyloxy midostaurin, said process comprising:

(a) reacting staurosporine with benzoic anhydride in a solvent comprising halogenated hydrocarbon or mixture of halogenated hydrocarbon & alcohol to produce midostaurin,

(b) isolating the midostaurin.

(c) optionally purifying the midostaurin obtained in step (b)

Step (a) involves reacting staurosporine with benzoic anhydride in a solvent comprising halogenated hydrocarbon or mixture of halogenated hydrocarbon & alcohol to produce midostaurin

The step (a) may be carried out at a temperature less than 100° C. or less than 80° C. or less than 60° C. or less than 40° C. or less than 20° C. or at the reflux temperature of the solvent or mixture of solvents used in step (a).

Step (b) involves isolating midostaurin.

Suitable isolation methods that may be used in step (b) include decantation or filtration or precipitation from a solvent or by removing the solvent or by concentrating the reaction mass or adding an anti-solvent to a solution or by evaporation of solution and the like or any other suitable isolation techniques known in the art. The said precipitation may result in a crystalline compound including solvates and hydrates thereof or amorphous form or essentially amorphous form. Suitable solvents that may be used for said isolation include water, alcohols, ketones, hydrocarbons, halogenated hydrocarbons, esters, ethers, polar aprotic solvents, nitriles or any mixtures thereof.

Purification of midostaurin obtained from step (b) may be carried out by one or more methods selected from slurrying in a solvent, recrystallization from a solvent or a chromatography.

Suitable solvents that may be used for purification of midostaurin by slurrying in a suitable solvent or recrystallization in a solvent include water, alcohols, ketones, hydrocarbons, halogenated hydrocarbons, esters, ethers, polar aprotic solvents, nitriles or any mixtures thereof.

Suitable chromatographic techniques that may be used for purification of midostaurin are selected from column chromatography, flash chromatography, ion exchange chromatography, supercritical fluid chromatography, high performance liquid chromatography (both reverse phase and normal phase), expanded bed adsorption chromatography and simulated moving bed chromatography or any combination thereof.

The purification process may be carried out one or more times using one or more purification methods described in the present application to completely remove the impurities or to get the desired purity of midostaurin.

In the eighth embodiment, the present application provides substantially pure midostaurin.

Following gradient HPLC method used to measure the purity of midostaurin and the content of impurities in midostaurin.

| Column | YMC Triart C18 | |
|---|---|---|
| Column Temperature | 35° | C. |
| Injection volume | 10 | μL |
| Diluent | Acetonitrile:Methanol | |
| Test concentration | 0.3 | mg/mL |

| | |
|---|---|
| Buffer | Ammonium acetate |
| Mobile Phase A | Mixture of buffer and Acetonitrile |
| Mobile Phase B | Mixture of water and Acetonitrile |

### Definitions

The following definitions are used in connection with the present application unless the context indicates otherwise. In general, the number of carbon atoms present in a given group or compound is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions or the like.

An “alcohol” is an organic compound containing a carbon bound to a hydroxyl group. “C₁-C₆ alcohols” include methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, cyclohexanol, phenol, glycerol and the like.

A “hydrocarbon solvent” is a liquid hydrocarbon compound, which may be linear, branched, or cyclic and may be saturated or have as many as two double bonds or aromatic. Examples of “C₅-C₁₅ aliphatic or aromatic hydrocarbons” include n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, petroleum ethers, benzene toluene, ethylbenzene, m-xylene, o-xylene, p-xylene, indane, naphthalene, tetralin, trimethylbenzene, chlorobenzene, fluorobenzene, trifluorotoluene, anisole, C₆-C₁₂ aromatic hydrocarbons and the like.

An “ether” is an organic compound containing an oxygen atom —O— bonded to two other carbon atoms. “C₂-C₆ ethers” include diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole and the like.

A “halogenated hydrocarbon” is an organic compound containing a carbon bound to a halogen. Halogenated hydrocarbons include dichloromethane, 1,2-dichloroethane, trichloroethylene, perchloroethylene, 1,1,1-trichloroethane, 1,1,2-trichloroethane, chloroform, carbon tetrachloride and the like.

An “ester” is an organic compound containing a carboxyl group —(C═O)—O-bonded to two other carbon atoms. “C₃-C₁₀ esters” include ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate and the like.

A “ketone” is an organic compound containing a carbonyl group —(C═O)— bonded to two other carbon atoms. “C₃-C₁₀ ketones” include acetone, ethyl methyl ketone, diethyl ketone, methyl isobutyl ketone, ketones and the like.

A “nitrile” is an organic compound containing a cyano —(C≡N) bonded to another carbon atom. “C₂-C₆ Nitriles” include acetonitrile, propionitrile, butanenitrile and the like.

A “polar aprotic solvents” include N, N-dimethylformamide, N, N-dimethylacetamide, dimethylsulfoxide, sulfolane, N-methylpyrrolidone and the like;

“Free from” as used herein refers to a compound that is having impurities below its limit of detection or not detected as measured by HPLC method or UPLC method or any other analytical method.

“Substantially free” as used herein refers to a compound that is having one or more individual impurities less than about 0.05% or less than about 0.02% or less than about 0.01% or less than about 0.001% or less than about 0.001% or less than about 0.0001% as measured by liquid chromatography method or any other analytical method.

“Substantially pure” as used herein refers to purity of the compound which is at least about 99.5% or at least about 99.6% or at least about 99.7% or at least about 99.8% or at least about 99.9% as measured by a liquid chromatography method or any other analytical method.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present application. While particular aspects of the present application have been illustrated and described, it would be obvious to those skilled in the art that various other changes and modifications can be made without departing from the spirit and scope of the invention. It is therefore intended to cover in the appended claims all such changes and modifications that are within the scope of this application.

### EXAMPLES

### Example-1: Preparation of Midostaurin

Benzoic anhydride (9.46 g) was added to the reaction mass containing staurosporine (15 g) and isopropyl alcohol (150 mL) at 26° C. The resultant reaction mixture was heated to 56° C. and stirred at 56° C. for 8 hours. Reaction mass filtered, washed with isopropyl alcohol (75 mL) and dried at 50° C. to afford title compound (17.5 g; Purity by HPLC: 99.1%, Staurosporine: 0.13%).

### Example-2: Preparation of Midostaurin

Benzoic anhydride (0.63 g) was added to the reaction mass containing staurosporine (1 g) and toluene (10 mL) at 26° C. The resultant reaction mixture was heated to 57° C. and stirred at 57° C. for 7 hours 30 minutes. Heptane (20 mL) was added to the reaction mass and stirred at 58° C. for 2 hours. Reaction mass filtered, washed with heptane and dried to afford title compound (Purity by HPLC: 99.0%, Staurosporine: 0.14%)

### Example-3: Preparation of Midostaurin

Benzoic anhydride (0.29 g) was added to the reaction mass containing staurosporine (0.5 g) and isopropyl acetate (15 mL) at 26° C. The resultant reaction mixture was heated to 83° C. and stirred at 83° C. for 5 hours. Reaction mass filtered, washed with isopropyl acetate to afford title compound (Purity by HPLC: 96.77% Staurosporine: 0.11%)

### Example-4: Preparation of Midostaurin

Benzoic anhydride (0.267 g) was added to the reaction mass containing staurosporine (0.5 g) and acetonitrile (15 mL) at 26° C. The resultant reaction mixture was heated to 81° C. and stirred at 83° C. for 12 hours. Reaction mass filtered, washed with acetonitrile to afford title compound (Purity by HPLC: 92.49% Staurosporine: 0.22%)

### Example-5: Preparation of Midostaurin Amorphous Form

Solution of Benzoic anhydride (6.3 g) in ethanol (30 mL) was added slowly to the reaction mass containing staurosporine (10 g) and ethanol (80 mL) at 26° C. The resultant reaction mixture was heated to 58° C. and stirred at 56° C. for 13 hours. Reaction mass filtered, washed with ethanol (50 mL) and dried at 50° C. to afford title compound (10.8 g; Purity by HPLC: 98.9%, Staurosporine: 0.72%)

### Example-6: Preparation of Midostaurin

Benzoic anhydride (0.776 g) was added slowly to the reaction mass containing staurosporine (1 g), dichloromethane (15 mL) and ethanol (15 mL) at 29° C. The resultant reaction mixture was stirred at 32° C. for 32 hours. Reaction mass filtered, washed with mixture of dichloromethane and ethanol (1:1; 3 mL). Filtrate solvent was distilled up to 14-16 volumes under atmospheric pressure at below 65° C. Reaction mass temperature increased to 75° C. and distilled up to 10-12 volumes at atmospheric pressure. Midostaurin crystalline Form II seed was added to the reaction mass and stirred at 75° C. for 15 minutes. Slowly cooled to ambient temperature and stirred at ambient temperature for 3 hours 30 minutes. Separated solid was filtered, washed with ethanol (4 mL) and dried at 125° C. till the individual solvents contents reaches to the acceptable limits according to ICH (1.14 g; Purity by HPLC: 99.9%, Staurosporine: 0.02%)

### Example-7: Preparation of Midostaurin

Benzoic anhydride (12.61 g) was added to the reaction mass containing staurosporine (20 g) and dichloromethane (200 mL) at 29° C. The resultant reaction mixture was stirred at 29° C. for 27 hours. Reaction mass concentrated up to 5 volumes at below 50° C., ethanol (200 mL) was added and concentrated up to 5 volumes at below 50° C. Ethanol (100 mL) was added to the reaction mass, temperature increased to 73° C. and stirred at 73° C. for 30 minutes. Midostaurin crystalline Form II seed was added to the reaction mass and stirred at 74° C. for 30 minutes. Slowly cooled to ambient temperature and stirred at ambient temperature for 15 hours. Separated solid was filtered, washed with ethanol (40 mL) and dried to afford title compound (24 g; Purity by HPLC: 99.9%, Staurosporine: 0.006%; palmitoyl staurosporine: Not detected; 7-oxo midostaurin: Not detected; hydroxy midostaurin: Not detected; 0-desmethyl midostaurin: Not detected, N-desmethyl midostaurin: Not detected, N-benzoyloxy midostaurin: Not detected;

### Example-8: Preparation of Midostaurin Crystalline Form-II

Benzoic anhydride (85 g) was added slowly to the reaction mass containing staurosporine (100 g), dichloromethane (1 L) and ethanol (3 L) at 26° C. The resultant reaction mixture was stirred at 40° C. for 35 hours. Reaction mass filtered, washed with mixture of dichloromethane and ethanol (1:1; 400 mL). Filtrate solvent was distilled up to 16-20 volumes under atmospheric pressure at below 65° C. Reaction mass temperature increased to 75° C. and distilled up to 10-12 volumes at atmospheric pressure. Midostaurin crystalline Form II seed was added to the reaction mass and stirred at 70° C. for 1 hour. Slowly cooled to ambient temperature and stirred at ambient temperature for 11 hours. Separated solid was filtered, washed with ethanol (500 mL). Wet compound charged into and ethanol (600 mL) charged in to round bottom flask and heated to 65° C. After stirring at 65° C. for 1 hour, cooled to ambient temperature and stirred for 1 hour. Filtered the reaction mass, washed with ethanol (100 mL) and dried at 125° C. to afford title compound

Purity by HPLC: 99.9%, Staurosporine: Not detected; palmitoyl staurosporine: Not detected; 7-oxo midostaurin: Not detected; hydroxy midostaurin: Not detected; O-desmethyl midostaurin: Not detected, N-desmethyl midostaurin: Not detected, N-benzoyloxy midostaurin: 0.02;

Ethanol content by GC: 3112 ppm; Dichloromethane content by GC: 398 ppm

1. A process for preparation of midostaurin, said process comprising reacting staurosporine with benzoic anhydride in a solvent comprising halogenated hydrocarbon or mixture of halogenated hydrocarbon & alcohol to produce midostaurin.

2. The process of claim 1, wherein the said process comprising:
(a) reacting staurosporine with benzoic anhydride in a solvent comprising mixture of halogenated hydrocarbon & alcohol to produce midostaurin,
(b) removing the halogenated hydrocarbon solvent,
(c) isolating the midostaurin, and
(d) optionally purifying the midostaurin obtained in step (c).

3. The process of claim 1, wherein the halogenated hydrocarbon solvent is dichloromethane.

4. The process of claim 1, wherein the alcohol solvent is C₁-C₂ alcohols.

5. The process of claim 1, wherein the alcohol solvent is ethanol.

6. The process of claim 1, wherein the solvent is mixture of dichloromethane and Ethanol.

7. The process of claim 1, further comprises drying midostaurin at about 120° C. to produce midostaurin having less than 5000 ppm of ethanol.

8. The process of claim 1, further comprises drying midostaurin at about 120° C. to produce midostaurin having less than 600 ppm of dichloromethane.

9. Midostaurin having less than 0.1% of one or more impurities selected from palmitoyl staurosporine, O-desmethyl midostaurin, N-desmethyl midostaurin, 7-oxo midostaurin, staurosporine, hydroxy midostaurin or N-benzoyloxy midostaurin.

10. The compound of claim 9, wherein midostaurin having less than 0.05% one or more impurities selected from palmitoyl staurosporine, O-desmethyl midostaurin, N-desmethyl midostaurin, 7-oxo midostaurin, staurosporine, hydroxy midostaurin or N-benzoyloxy midostaurin.

11. The compound of claim 9, wherein midostaurin having less than 0.01% one or more impurities selected from palmitoyl staurosporine, O-desmethyl midostaurin, N-desmethyl midostaurin, 7-oxo midostaurin, staurosporine, hydroxy midostaurin or N-benzoyloxy midostaurin.

12. The compound of claim 9, midostaurin free of one or more impurities selected from palmitoyl staurosporine, O-desmethyl midostaurin, N-desmethyl midostaurin or 7-oxo midostaurin.

13. The compound of claim 9, midostaurin substantially free of one or more impurities selected from staurosporine, hydroxy midostaurin or N-benzoyloxy midostaurin.

14. The process for preparation of midostaurin of claim 9, wherein said process comprising:
(a) reacting staurosporine with benzoic anhydride in a solvent comprising halogenated hydrocarbon or mixture of halogenated hydrocarbon & alcohol to produce midostaurin,
(b) isolating the midostaurin.
(c) optionally purifying the midostaurin obtained in step (b)

15. The process of claim 14, wherein the solvent is dichloromethane or mixture of dichloromethane and ethanol.
